(19) United States
(12) Patent Application Publication
(10) Pub. No.: US 2025/0002566 A1
(43) Pub. Date: Jan. 2, 2025

(54) POLYNUCLEOTIDE, EXPRESSION CASSETTE, VECTOR, HOST CELL, POLYPEPTIDE, PHARMACEUTICAL COMPOSITION, USE OF A POLYPEPTIDE, METHOD FOR PRODUCING A POLYPEPTIDE, AND METHOD FOR PREVENTING OR TREATING INFECTIONS CAUSED BY FLAVIVIRUSES

(71) Applicant: FUNDAÇÃO OSWALDO CRUZ, RIO DE JANEIRO (BR)

(72) Inventors: Roberto Dias Lins Neto, Recife (BR); Carlos Henrique Bezerra Da Cruz, Olinda (BR); Isabelle Freire Tabosa Viana, Recife (BR); Margarida Archer Baltazar Pereira Da Silva Franco Frazão, Oeiras (PT); Diogo Pinto Pereira De Athayde, Odivelas (PT)

(21) Appl. No.: 18/750,516

(22) Filed: Jun. 21, 2024

(30) Foreign Application Priority Data
Jun. 22, 2023 (BR) 10 2023 012638 3

(51) Int. Cl. C07K 16/10

(52) U.S. Cl. CPC C07K 16/1081; C07K 2317/76; C07K 2317/92

(57) ABSTRACT

The development of therapeutics against the Zika virus (ZIKV) requires the design of molecules capable of neutralizing the virus and preventing cellular infection. Among the known ZIKV epitopes for neutralizing antibodies, the fusion loop (FL), located on the envelope (E) protein, is of particular interest as it mediates the first step of cellular infection and is highly conserved among flaviviruses. Here, small synthetic proteins were computationally designed to bind ZIKV FL with high affinity. The candidate with the highest predicted affinity was synthesized experimentally. It binds to its target epitope with high affinity, either in the context of the E protein or within the entire virus. The protein also showed cross-reactive neutralization capacity against ZIKV and Dengue virus 1-2 in vitro. X-ray crystallography was used to validate the computational design as well as to provide additional insights into the structural basis of flavivirus neutralization by targeting the FL envelope protein.

### SEQUENCE LISTING

The instant application contains a Sequence Listing which has been submitted electronically in XML format and is hereby incorporated by reference in its entirety. Said XML file, created on Jun. 18, 2024, is named PE003483 Sequence Listing.xml and is 4,591 bytes in size.

### FIELD

This application claims the benefit of priority to Brazilian Patent Application No. BR 10 2023 012638 3, filed on Jun. 22, 2023, which entire contents are incorporated herein by reference.

The present invention relates to the field of immunology and biotechnology. In particular, the present invention relates to polypeptides useful in the prevention and treatment of flavivirus-related conditions.

### BACKGROUND

Zika virus (ZIKV) is a member of the Flavivirus genus, which includes Dengue Virus (DENV), Yellow Fever Virus (YFV) and West Nile Virus (WNV), among others highly transmissible by mosquito bites [1]. Viruses of this genus are known to cause a wide range of clinical symptoms varying from mild illness to severe and fatal hemorrhagic and neurological conditions [2-4]. Although ZIKV has long been associated with mild and sporadic infections in Africa [5, 6], this virus was responsible for the most devastating arboviral epidemic in recent decades when it arrived in Brazil in 2015 [7]. ZIKV has already achieved worldwide distribution and is associated with several critical outcomes, including Guillain-Barré and Zika congenital syndrome (ZCS) [8, 9]. Unlike other flaviviruses, ZIKV has been found to persist in body fluids for up to six months and is associated with sexual and transplacental transmission [10]. In the absence of licensed vaccines or therapies, treatment of symptoms and infection control remain the most effective ways of combating the disease.

Like other flaviviruses, ZIKV has a non-segmented, single-stranded, positive-sense enveloped RNA that encodes 10 viral proteins, including 3 structural proteins (envelope-E, capsid-C and pre-membrane-prM) and seven non-structural proteins associated with viral replication (NS1, NS2A, NS2B, NS3, NS4A, NS4B and NS5) [11]. The viral surface is composed of 180 copies of E proteins arranged in 90 antiparallel dimers and distributed in a herringbone configuration [12, 13]. Each E protein folds into a rod-like structure and is composed of three β-sheet-rich domains (DI, DII and DIII). Between them, DI and DII form an elongated finger-like structure that expands distally into a highly hydrophobic, glycine-rich fusion loop (FL), which is conserved among flaviviruses and mediates cell infection [14, 15].

Protein E is the main target of the antibody response against ZIKV [16]. Although several neutralizing epitopes have been identified in all three E domains [17], the immune response after infection is mainly dominated by antibodies targeting DI and DII. Among these antibodies, most target the FL and are known to exhibit broad cross-reactivity and varying degrees of neutralizing activity. Although considered less potent than antibodies against DIII, previous reports have shown the protective efficacy of anti-FL antibodies against flaviviruses in vivo [18-21]. Other studies have shown that the frequency of human neutralizing antibodies within the overall polyclonal response is higher against FL within DII than against DIII. These antibodies have been shown to provide a higher and broader anti-ZIKV neutralizing capacity that protects mice against African and Asian-American strains in non-pregnant and pregnant mouse models, preventing maternal-fetal transmission, dam infections and disease [22].

Most antibodies against LF are sensitive to small changes in the primary sequence of the target epitope [23, 24], and the interaction at the interface has remained poorly understood. The crystal structure of the E protein complexed with the broadly flavivirus-protective murine antibody 2A10G6 has recently become available [25]. This antibody was previously shown to neutralize ZIKV, DENV1-4, YFV and WNV in vitro [19]. The solved structure of this complex showed a discrete amphiphilic interface, with W101 and F108 in the FL epitope performing strong hydrophobic interactions with Y93 of the antibody's L chain and persistent contacts with H40, N64 and R104 of the H chain.

Several attempts have been made to prepare vaccines and compounds that are effective against flaviviruses in general and ZIKV in particular.

França et. al., 2022 presents a method for constructing neutralizing recombinant antibodies to the Zika virus. The antibody construction process starts from the mimetic peptide based on the fusion loop (FL) region in the E protein of the Zika virus.

EP4056582A aims to provide a vaccine against Zika/Dengue and its application to prevent the ADE effect. The document obtained the epitope data of an antibody that causes the ADE effect using crystalline structure analysis and other structural and functional analyses. Thus, antigens are provided in the document, for which some mutations are introduced into the FL fusion region of the E protein of a Zika virus or a Dengue virus. The antigens with the mentioned mutations are unable to bind to the antibodies that cause ADE (FLE antibody). An embodiment of the document also provides a vaccine, which can prevent the production of antibodies induced by the FL epitope after immunization, thus reducing or eliminating the ADE effect.

WO2017212291A1 for its part, discloses methods, compositions and use as a vaccine based on a region of the E protein called the E dimer epitope (EDE), which indicates the ability to neutralize infections from flaviviruses such as Zika and Dengue. In one embodiment, the invention provides, for example, an isolated neutralizing antibody directed against the EDE, optionally in which said antibody, or a fragment thereof binds to the five polypeptide segments of the ectodomain (SE) of the E glycoprotein of the dengue virus, optionally in which the binding is not affected by the presence or absence of dengue glycan N153 (Zika N154) or corresponding residue.

As noted, however, in EP4056582A, there is substantial evidence showing that pre-existing antibodies after ZIKV infection can enhance a subsequent DENV infection due to cross-reactivity with DENV (Fowler et al., 2018; George et al., 2017: Li et al., 2017; Richner et al., 2017; al., 2017; Stettler et al., 2016; Valiant et al., 2018). This phenomenon is called antibody-dependent enhancement (ADE). ADE refers to an antibody that increases viral infection when the antibody is insufficient to neutralize the virus or at a sub-neutralizing concentration (Beltramello et al., 2010; Dejnirattisai et al., 2010). Although epidemiological investigations are still insufficient, pre-existing ZIKV antibodies from humans, monkeys and mice have been shown to increase DENV infection in cell experiments (George et al., 2017; Richner et al., 2017; Stettler et al., 2016; Valiant et al., 2018). In addition, it has been confirmed in monkey and mouse models that the symptoms of DENV infection can be exacerbated by antibodies obtained from a ZIKV infection, immunization by vaccine or in a fetus by its mother (Fowler et al., 2018; George et al., 2017; Richner et al., 2017; Stettler et al., 2016). Therefore, it should be considered that a ZIKV vaccine may have an ADE effect on a future DENV infection after immunization.

Thus, there is a need to develop compounds that are effective in treating and/or preventing ZIKV infections, but at the same time do not lead to an ADE effect in possible future infections by other flaviviruses.

### SUMMARY OF THE INVENTION

In a first aspect, the present invention refers to a polynucleotide selected from the group consisting of:
(a) polynucleotides comprising a nucleotides sequence as set out in SEQ ID NO: 1;
(b) nucleic acids that hybridize under stringent conditions with the nucleic acid of SEQ ID NO: 1;
(c) polynucleotides that encode a polypeptide that is at least 70% identical to the amino acid sequence of SEQ ID NO: 2 or 3;
(d) degenerated sequences of polynucleotides from (a)-(c).

In a preferred embodiment, the polynucleotide is a cDNA, genomic DNA, synthetic DNA or RNA.

In a second aspect, the present invention refers to an expression cassette comprising a polynucleotide according to the first aspect of the invention, operably linked to a promoter and to a transcription terminator.

In a third aspect, the invention refers to an expression vector, comprising the polynucleotide according to the first aspect of the invention, or an expression cassette according to the second aspect of the invention.

In a fourth aspect, the invention refers to a host cell, comprising the polynucleotide according to the first aspect of the invention, the expression cassette according to the second aspect of the invention or the expression vector according to the third aspect of the invention.

In a fifth aspect, the invention refers to a polypeptide, selected from the group consisting of:
(a) a polypeptide comprising the amino acid sequence as set out in SEQ ID NO: 2 or 3:
(b) a polypeptide comprising an amino acid sequence having at least 70% identity with the sequence as set out in SEQ ID NO: 2 or 3;
in its pure form, or fused or complexed with nanomaterials, hydrogels, other proteins, liposomes and any other carrier and/or release vehicle.

In a sixth aspect, the invention refers to a pharmaceutical composition comprising a polypeptide according to the fifth aspect of the invention, and a pharmaceutically acceptable carrier or excipient.

In a preferred embodiment, the composition is a medicine, a vaccine or a therapeutic vaccine.

In another preferred embodiment, the polypeptide according to the fifth aspect of the invention, or the pharmaceutical composition according to the sixth aspect of the invention, are for the prevention or treatment of infections caused by flaviviruses.

In an even more preferred embodiment, the flaviviruses are selected from Zika Virus (ZIKV) or Dengue Virus (DENV-1, DENV-2).

In a seventh aspect, the invention refers to the use of the polypeptide according to the fifth aspect of the invention, for the manufacture of a medicine for the prevention or treatment of infections caused by flaviviruses.

In a preferred embodiment, the flaviviruses are selected from Zika Virus (ZIKV) or Dengue Virus (DENV-1, DENV-2).

In an eighth aspect, the invention refers to a method for producing a polypeptide, comprising:
(a) providing a host cell according to the fourth aspect of the invention:
(b) culturing said cell under conditions conducive to the production of the polypeptide; and
(c) isolating said polypeptide from said cell or the culture medium in which it is inserted.

In a last aspect, the invention refers to a method for preventing or treating infections caused by flaviviruses, comprising administering a therapeutically effective amount of the polypeptide according to the fifth aspect of the invention, or the pharmaceutical composition according to the sixth aspect of the invention to a subject in need thereof.

In a preferred embodiment, the flavivirus are selected from Zika Virus (ZIKV) or Dengue Virus (DENV-1, DENV-2).

### BRIEF DESCRIPTION OF THE FIGURES

FIG. 1. (A) The ZIKV envelope (E) protein complexed to ZVPA3 is represented on transparent surfaces, where its main chains are represented as spirals (α-helix) and arrows (β-sheet). The three projected loops of ZVPA3 are highlighted by “Loop 1”, “Loop 2” and “Loop 3”. (B) The main interacting residues between the designed protein and ZIKV FL are shown. The dotted line along the x-axis represents the boundary between the two proteins. Electrostatic interactions are shown by dotted lines between residues along the y-axis associated with their inter-residue distances in Angstroms (Å), which are represented by ball and stick models. Residues involved in hydrophobic contacts are shown as semicircular lines facing their interaction partner at the interface.

FIG. 2. Biophysical characterization of ZVPA3. (A) CD spectrum of ZVPA3 purified at 0.25 mg/mL in 20 mM Na/Na Phosphate at pH 8.0. (B) NanoDSF curves of intrinsic fluorescence at 330 nm and its first derivative of ZVPA3 at 0.25 mg/mL in different buffers. The vertical lines represent the melting temperature (Tₘ) of the protein.

FIG. 3. X-ray structure of ZVPA3. (A) The asymmetric crystalline unit shows two protein monomers (as shown in FIG. 3A), represented as drawings and colored by the B-factor ramp. Darker shades of gray represent regions of low values while lighter shades of gray represent regions of high values. The contact interface of the Loop3 (V141-P156) A-chain with the B-chain is shown with the relevant residues labeled and highlighted in the inset, in which the secondary structure is represented by arrows for β-sheet and spirals for α-helix: the residues are shown as sticks. (B) A-chain represented as a drawing, where spirals represent α-helix and arrows the β-sheet.

FIG. 4. Superposition of the computer-designed protein structure (dark gray) over the structure determined by crystallography (light gray), shown as design models. (A) Superposition of the computer model on the crystalline structure of the A chain of ZVPA. (B) Superposition of the computer model on the crystalline structure of the B chain of ZVPA. (The structures in FIGS. 4A and 4B are shown in the same orientation as in FIGS. 1A and 3B.) (C) Computer-generated and crystalline structures (from FIG. 4B) are shown rotated by 90° on their vertical (y) axis, showing the binding pocket and the atomic positions of all residues that come into contact with the ZIKV FL within 3.0 A in the designed protein (as in FIG. 1B) as a rod model.

FIG. 5. ZVPA3 binding and virus neutralization activity. (A e B) Comparison of ZVPA3 binding curves between the target epitope (ZIKV FL) in the context of the isolated ZIKV E protein (A) and in the context of the entire ZIKV particle (B). (Error bars represent standard deviations of three individual repeated experiments). (C) Evaluation of the neutralization activity of ZIKV (curve with circular dot) and DENV1 (curve with square dot) and DENV2 (curve with triangular dot) by PRNT. (Each curve was run in duplicate, and the data is shown as the median plus the standard deviation). A t-test was performed and showed no significant difference between the groups tested (p-value=0).7251), indicating similar neutralization efficacy of ZVPA3 for ZIKV. DENV1 and DENV-2.

FIG. 6. ZVPA3 production. (A) SEC chromatogram of ZVPA3 after purification by affinity chromatography. UV absorbance at 280 nm (mAU) was monitored over time and protein fractions were collected. (B) 15% SDS-PAGE of SEC eluted fractions showing a predominant band in all lanes around 20 kDa. The molecular weight marker (MWM) used was PageRuler™ Plus Prestained Protein Ladder (ThermoFisher Scientific).

FIGS. 7A, 7B, and 7C, collectively FIG. 7. (A) Data collection and refinement statistics for the ZVPA3 protein. The statistics for the highest resolution shell are shown in brackets. 5% of the reflections were used to calculate the Rfree. (B) ZVPA3 crystals and (C) X-ray diffraction pattern.

FIG. 8. Assessment of flavivirus neutralization in vitro. (A) Assessment of ZIKV neutralization activity (dark grey curve) by the microneutralization test. The assay was carried out in triplicate and the data is presented as the median plus the standard deviation. The ZVPA3 protein was used as a control (light gray curve) and does not bind nonspecifically to the antibodies used to develop the assay. Dotted lines at 0 and 100 are shown for clarity. PRNT plates showing neutralization against ZIKV (B), DENV-1 (C) and DENV-2 (D) are also shown. Highlighted in dark gray and white outline and font are the negative and positive control wells, respectively. The ZVPA3 dilution curve is shown in light gray on each plate.

### DETAILED DESCRIPTION

The following definitions serve to provide a clear and consistent understanding of the specification and claims, which include the scope to be given such terms.

In one aspect, the present invention refers to a polynucleotide selected from the group consisting of:
(a) polynucleotides comprising a nucleotides sequence as set out in SEQ ID NO: 1:
(b) nucleic acids that hybridize under stringent conditions with the nucleic acid of SEQ ID NO: 1:
(c) polynucleotides that encode a polypeptide that is at least 70% identical to the amino acid sequence of SEQ ID NO: 2 or 3:
(d) degenerated sequences of polynucleotides from (a)-(c).

In a preferred embodiment, the polynucleotide is a cDNA, genomic DNA, synthetic DNA or RNA.

The terms “nucleic acid” and “polynucleotide” are used interchangeably and refer to RNA and DNA. The polynucleotides can be single or double stranded. Non-limiting examples of polynucleotides include genes, gene fragments, exons, introns, messenger RNA, siRNA, miRNA, complementary DNA, genomic DNA, synthetic DNA, recombinant DNA, cassettes, vectors, probes and primers. The term “recombinant DNA” refers to any artificial nucleotide sequence that results from the combination of DNA sequences from different sources.

The term “degenerated nucleotide sequence” denotes a nucleotide sequence that includes one or more degenerated codons when compared to a reference nucleic acid molecule that codes for a particular polypeptide. Degenerated codons contain different nucleotide triplets but encode the same amino acid residue (e.g. GAU and GAC both encode Asp).

In another aspect, the invention refers to a polypeptide selected from the group consisting of:
(a) a polypeptide comprising the amino acid sequence as set out in SEQ ID NO: 2 or 3:
(b) a polypeptide comprising an amino acid sequence having at least 70% identity with the sequence as set out in SEQ ID No: 2 or 3:
in its pure form, or fused or complexed with nanomaterials, hydrogels, other proteins, liposomes and any other carrier and/or release vehicle.

The term “peptide” as used herein means a compound which is manufactured from two or more amino acids linked by covalent bonds which are formed by the elimination of an H₂O molecule from the amino group of one amino acid and the carboxyl group of the next amino acid. In the present application, the terms “peptide”, “polypeptide” or “protein” can be used interchangeably and refer to an amino acids polymer linked by peptide bonds, regardless of the number of amino acid residues making up this chain. The polypeptides, as used herein, include “variants” or “derivatives” thereof, which refer to a polypeptide that includes variations or modifications, for example, substitution, deletion, addition or chemical modifications in its amino acid sequence in relation to the reference polypeptide, provided that the derived polypeptide shows the desired activity, stability, half-life, pharmacokinetic characteristics and/or physicochemical characteristics equal to or superior to those initially observed for the original polypeptide. Examples of chemical modifications are glycosylation, PEGlation, PEG alkylation, alkylation, phosphorylation, acetylation, amidation, etc. The amino acids of the polypeptides of the invention, depending on the orientation of the amino group of the alpha carbon atom can belong to the L or D series.

The polypeptide can be artificially manufactured from nucleotide sequences cloned through the recombinant DNA technique (“recombinant polypeptide”) or can be manufactured through a known chemical synthesis reaction (“synthetic polypeptide”), or by any techniques currently available and recognized by experts in the field.

The term “amino acid substitutions” refers to the replacement of at least one amino acid residue of polypeptides in order to produce derivatives with activity, stability, half-life, pharmacokinetic characteristics and/or physicochemical characteristics equal to or greater than those initially observed for the original polypeptides. The amino acid substitutes can be natural, modified or unusual.

In this respect, the term “conservative amino acid substitution” refers to the substitution of amino acids in a polypeptide with those with similar side chains and therefore very similar physicochemical properties. For example, the exchange of an alanine for a valine or leucine or isoleucine is considered conservative, since the amino acids involved have an aliphatic side chain as a common characteristic. The group containing a basic side chain comprises lysine, arginine and histidine. The group containing sulfur in the side chain includes the amino acids cysteine and methionine. The amino acids phenylalanine, tyrosine and tryptophan each have an aromatic side chain. Asparagine and glutamine are part of the amino acids with an amide side chain, while serine and threonine each have a hydroxyl linked to their aliphatic side chain. Other examples of conservative substitution include the substitution of a nonpolar or hydrophobic amino acid such as isoleucine, valine, leucine or methionine for another that is also nonpolar. Similarly, the invention herein described contemplates the substitution of polar or hydrophilic amino acids such as arginine for lysine, glutamine for asparagine and threonine for serine. In addition, substitution between basic amino acids such as lysine, arginine or histidine or substitution between acidic amino acids such as aspartic acid or glutamic acid is also contemplated. Examples of conservative amino acid substitutions are valine for leucine or isoleucine, phenylalanine for tyrosine, lysine for arginine, alanine for valine and asparagine for glutamine. In this invention, substitution matrices used in the amino acid alignment of proteins such as BLOSUM62 can also be used to determine which amino acids are most likely to replace another residue in a particular peptide sequence (Henikoff & Henikoff. 1992 PNAS, 89:10915-10919).

In addition, illustrative examples of modified or unusual amino acids include 2-aminoadipic acid, 3-aminoadipic acid, beta-alanine, 2-aminobutyric acid, 4-aminobutyric acid, 6-aminocaproic acid, 2-aminoheptanoic acid, 2-aminoisobutyric acid, 3-aminoisobutyric acid, 2-aminoisobutyric acid, 2-aminoheptanoic acid, 2-aminopimelic acid, 2,4-diaminobutyric acid, desmosine, 2,2′-diaminopimelic acid, 2,3-diaminopropionic acid, N-ethyl glycine, N-ethyl asparagine, hydroxylysine, allo-hydroxylysine, 3-hydroxyproline, 4-hydroxyproline, isodesmosine, alloisoleucine, N-methyl glycine, N-methyl isoleucine, 6-N-methyl-lysine, N-methyl-valine, norvaline, norleucine, ornithine, etc.

The phrase “which corresponds to” is used here to refer to similar or homologous sequences, whether the exact position is identical to or different from the molecule to which the similarity or homology is measured. A nucleic acid or amino acid sequence alignment can include gaps. Thus, the term “which corresponds to” refers to sequence similarity, and not to the numbering of amino acid residues or nucleotide bases.

The term “derivative(s)” A peptide as used herein refers to a peptide comprising variations of, for example, modification, substitution, deletion and/or addition in its amino acid sequence, made with respect to a reference peptide, which retain an identifiable relationship to the reference peptide. The modification may include chemical modification, for example, by glycosylation, PEGlation, PEG alkylation, alkylation, acetylation, amidation, glycosyl-phosphatidyl inositization, farnesylation, ADP-ribosylation, sulfation, lipid binding, hydroxy lation, and/or phosphorylation. This term is understood to be interchangeable with the term “functional analog”.

The term “fragment” refers to a specific region of the nucleotide or polypeptide sequence corresponding to the sequences indicated in this invention which exert the desired immunosuppressive function.

The term “identity” is defined as the level of equality between DNA or amino acid sequences when compared nucleotide by nucleotide or amino acid by amino acid with a reference sequence.

The term “similarity” is defined as the level of equality between two or more DNA or amino acid sequences when compared nucleotide by nucleotide or amino acid by amino acid.

The term “sequence identity percentage” refers to comparisons between polynucleotides or polypeptides and is determined by two ideally aligned sequences, under certain comparison parameters. This alignment can include gaps, generating intervals when compared to the reference sequence, which facilitate a proper comparison of the two. In general, the calculation of the identity percentage considers the number of positions where the same nucleotide or amino acid occurs in the sequences compared to the reference sequence, and is carried out using various sequence comparison algorithms and programs known in the state of the art. These algorithms and programs include, but are not limited to, TBLASTN, BLASTP, FASTA, TFASTA, CLUSTALW, FASTDB.

Similarity is estimated in this invention using comparison methods and parameters equivalent to those used to estimate the percentage of sequence identity, without, however, making the comparison in relation to a reference sequence. To estimate the percentage of similarity, comparisons between polynucleotides or polypeptides are made between two or more ideally aligned sequences.

For the purposes of the present invention, the term “complementary” is defined as the ability of the sense strand (5′→3′ direction) of one nucleotide segment to hybridize with an antisense strand (3′→5′ direction) of another nucleotide segment, under appropriate conditions, to form a double helix.

In another aspect, the present invention refers to an expression cassette comprising a polynucleotide according to the invention, operably linked to a promoter and to a transcription terminator.

An “expression cassette” refers to a nucleic acid construct comprising a coding region and a regulatory region, operably linked which, when introduced into a host cell, results in the transcription and/or translation of an RNA or polypeptide, respectively. Generally, an expression cassette comprises or consists of a promoter which enables transcription to be initiated, a nucleic acid according to the invention, and a transcription terminator. The expression “operably linked” indicates that the elements are combined so that the expression of the coding sequence is under the control of the transcriptional promoter and/or signal peptide. Typically, the promoter sequence is placed upstream of the gene of interest, at a distance from it that is compatible with controlling expression. Similarly, the signal peptide sequence is usually fused upstream of the sequence of the gene of interest, in phase with it, and downstream of any promoter. Spacing sequences may be present between the regulatory elements and the gene, although they do not prevent expression and/or screening. In one embodiment, said expression cassette comprises at least one enhancer sequence operably linked to the promoter.

In another aspect, the invention refers to an expression vector, comprising the polynucleotide, or an expression cassette according to the invention.

The term “vector” refers to nucleic acid molecules designed to transport, transfer and/or store genetic material, as well as express and/or integrate the genetic material into the chromosomal DNA of the host cell, such as plasmids, cosmids, artificial chromosomes, bacteriophages and other viruses. The vector usually consists of at least three basic units, the replication origin, a selection marker and the multiple cloning site.

The vectors used in this invention preferably have at least one “selection marker”, which is a genetic element that allows the selection of genetically modified organisms/cells. These markers include antibiotic resistance genes such as, but not limited to ampicillin, chloramphenicol, tetracycline, kanamycin, hygromycin, bleomycin, fleomycin, puromycin and/or phenotype complementation genes such as, but not limited to methotrexate, dihydrofolate reductase, ampicillin, neomycin, mycophenolic acid, glutamine synthetase.

The term “expression vector” refers to any vector that is capable of transporting, transferring and/or storing genetic material, and which, once in the host cell, is used as a source of genetic information to produce one or more gene products (gene expression).

In addition, the expression vectors of this invention may include one or more regulatory nucleotide sequences for controlling gene replication, transfer, transport, storage and expression of genetic material, such as origin of replication, selection marker, multiple cloning site, promoter (e.g. T7 pol, pL and lambda pR phage, SV40, CMV, HSV tk, pgk, T4 pol, or alpha EF-1 and its derivatives), ribosome binding site, RNA splice site, polyadenylation site, signal peptide for secretion and gene transcription termination sequence. However, the expression vectors of this invention are not limited to these. The technique for incorporating the control sequences into a vector is well characterized in the state of the art.

The expression vector used in this invention may also have “enhancer” sequences, also called “cis” elements that can positively or negatively influence promoter-dependent gene expression.

Furthermore, the expression vector used can be purchased commercially from among those that are commonly available and suitable. For example, the expression vector could be pET14b supplied by Genscript, with the following characteristics: Expression system: Bacterial; Promoter: T7; Expression level: High; Size: 5671 base pairs (bp): Antibiotic resistance: Ampicillin; Expression tag: Target protein is produced fused to a 6-histidine tail (present in the vector).

Thus, in one embodiment of the invention, the protein, as produced, has a histidine tail, as well as other amino acids from the expression vector, as shown below by the sequence as established by SEQ ID NO. 3:

MGSSHHHHHHSSGLVPRGSHMAKGDPHVLLTTSAGNIELELDSQKAPV
SVQNFLDYVNNGFYNNTTFHSVIPGYMIQGGGYTEDMQQKQPNPPIKN
EADNGLRNTRGTIAMMRTEDKDSATSQFFINVADNAYADHGQTRYGFA
VFGKVVKGMDVADKISQVPTHDVGPYQSVPSKPVVILSATVLPGSGC

in which the underlined amino acids correspond to the protein of interest, corresponding to SEQ ID NO. 2, and the other amino acids are derived from the commercial vector in which the protein can be produced.

A “coding sequence” refers to a nucleotide sequence that is transcribed into mRNA (messenger RNA) and translated into a polypeptide when under the control of appropriate regulatory sequences. The boundaries of the coding sequence are determined by a translation initiation codon at the 5′ end of the DNA sense strand and a translation termination codon at the 3′ end of the DNA sense strand. As a result of the degeneration of the genetic code, different DNA sequences can code for the same polypeptide sequence. It is therefore believed that these degenerated substitutions in the coding region are inserted in the sequences described in this invention.

The term “promotor” is a minimum DNA sequence sufficient to target gene transcription, i.e. a sequence that targets the binding of the RNA polymerase enzyme, thus promoting the synthesis of messenger RNA. Promoters can be specific to the type of cell, type of tissue and species, as well as being modulated, in certain cases, by regulatory elements in response to some external physical or chemical agent called an inducer.

The terms “transformation” and “transfection” refer to the act of inserting a vector or other vehicle carrying exogenous genetic material into a host cell, prokaryotic or eukaryotic, for transportation, transfer, storage and/or gene expression of the genetic material of interest.

The term “recombinant expression” refers to the expression of the recombinant polypeptide in host cells.

In another aspect, the invention refers to a host cell, comprising the polynucleotide according to the first aspect of the invention, the expression cassette according to the second aspect of the invention or the expression vector according to the third aspect of the invention.

The term “host cell” refers to the cell that will receive the genetic material through a vector and/or cells that have already received the genetic material through a vector (transformed or transfected cells). These host cells can be either prokaryotic (prokaryotic microorganisms) or eukaryotic (eukaryotic cells or microorganisms).

In another aspect, the invention refers to a method for producing a polypeptide comprising:
(a) providing a host cell according to the fourth aspect of the invention:
(b) culturing said cell under conditions conducive to the production of the polypeptide; and
(c) isolating said polypeptide from said cell or the culture medium in which it is inserted.

In another aspect, the invention refers to a pharmaceutical composition comprising a polypeptide according to the fifth aspect of the invention, and a pharmaceutically acceptable carrier or excipient.

As used herein, the terms “carrier”, “excipient” or “diluents” refer to any component of a pharmaceutical composition which is not the drug substance. Consequently, the term “pharmaceutically acceptable carrier” refers to a non-toxic, inert, solid, semi-solid or liquid filler, diluent, encapsulation material or formulation aid of any kind, or simply a sterile aqueous medium such as saline solution. Some examples of materials that can serve as pharmaceutically acceptable carriers are sugars such as lactose, glucose and sucrose, starches such as corn starch and potato starch, cellulose and its derivatives such as sodium carboxymethyl cellulose, ethyl cellulose and cellulose acetate; tragacanth powder; malt, gelatine, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline solution, Ringer's solution; ethyl alcohol and phosphate buffer solutions, as well as other non-toxic compatible substances used in pharmaceutical formulations.

In a preferred embodiment, the composition is a medicine, a vaccine or a therapeutic vaccine.

The term “vaccine”, as used herein, refer to a pharmaceutical composition which comprises immunogenic components capable of inducing a specific immune response against an infectious agent, such as a virus. This immune response can involve the production of antibodies, specialized immune cells or a combination of both. The vaccine can be administered to prevent infection by the pathogen, reduce the severity of symptoms associated with the infection or induce long-term protective immunity against future exposure to the infectious agent. In addition, the vaccine may contain adjuvants or other ingredients that enhance the immune response, as well as formulation components that ensure the stability and efficacy of the vaccine during storage and administration.

As used herein, the term “therapeutic vaccine” refers to a pharmaceutical composition which comprises immunogenic components designed to treat or modulate an existing health condition in a subject. The therapeutic vaccine can be designed to induce a specific immune response against antigens associated with the health condition in question, with the aim of eliminating, reducing the progression of or controlling the symptoms of the disease. This immune response may involve the activation of specific immune cells, the production of antibodies or a combination of both. In addition, the therapeutic vaccine may contain adjuvants, immunostimulant molecules or other technologies that enhance the immune response and improve the effectiveness of the treatment. The therapeutic vaccine can be administered by various routes, with the aim of providing the desired immune response at the site affected by the health condition in question.

In another preferred embodiment, the polypeptide, or the pharmaceutical composition according to the invention, are for the prevention or treatment of infections caused by flaviviruses.

In another aspect, the invention refers to the use of the polypeptide according to the invention, for the manufacture of a drug for the prevention or treatment of infections caused by flaviviruses.

In a last aspect, the invention refers to a method for preventing or treating infections caused by flaviviruses, comprising administering a therapeutically effective amount of the polypeptide according to the fifth aspect of the invention, or the pharmaceutical composition according to the sixth aspect of the invention to a subject in need thereof.

As used herein, the term “flavivirus” refers to any virus pertaining to the Flaviviridae family. Preferably, the flavivirus according to the present invention encompass several viral agents transmitted by arthropods. Flaviviruses also include important human and veterinary pathogens, such as dengue virus, yellow fever virus, Japanese encephalitis virus, West Nile virus and Zika virus.

According to the invention, the active agent is preferably administered in an effective amount. As used herein, the phrase “effective amount” refers to the amount of a component that is sufficient to produce a desired therapeutic response without undue adverse side effects (such as toxicity, irritation or allergic response) commensurate with a reasonable benefit/risk ratio when used in the manner presently described. For example, a “therapeutically effective amount” may be a sufficient amount of the active agent to cause regression of the viral infection, or at least partially halt its progression and spread, in a reasonable benefit/risk ratio applicable to any medical treatment. The actual amount administered, as well as the rate and duration of administration, will depend on the nature and severity of the condition being treated. The prescription of treatment, including decisions on dosage, timing, etc., is the responsibility of qualified professionals and specialists, taking into account the disorder being treated, the patient's individual condition, the site of action, the method of administration and other factors known to the professionals.

As used herein, the terms “administering,” “administration,” and similar terms refer to any method which, in judicious medical practice, releases the composition or compound presently supplied to a subject in such a manner as to provide a therapeutic effect. One aspect of the present invention provides parenteral administration of a therapeutically effective amount of the present composition to a subject in need thereof. Yet, another aspect of the present invention provides enteral administration of a therapeutically effective amount of the present composition to a subject in need thereof.

As used herein, the term “treating” includes the prophylaxis of a viral infection in a patient or subject with a tendency to develop such a viral infection, and the improvement or elimination of the developed viral infection once it has been established or relief of the symptoms characteristic of that viral infection. In a preferred embodiment, the infection is caused by flaviviruses. In an even more preferred embodiment, flaviviruses are selected from Zika Virus (ZIKV) or Dengue Virus (DENV-1, DENV-2).

As used herein, the term “subject” refers to any subject, particularly a mammalian subject for which diagnosis, prognosis, or therapy is desired, for example, a human being. The term can be used to refer to a “patient.”

Unless otherwise defined, all the technical and scientific terms used herein have the same meaning as usually understood by a person of ordinary skill in the art to which the matter presently described belongs.

Throughout the application, descriptions of various embodiments use the term “comprising,” that will be understood by a person skilled in the art that in some specific cases, an embodiment can alternatively be described using the language “consisting essentially of” or “consisting of.”

For the purposes of a better understanding of the present disclosures and in no way limiting the scope of the disclosures, unless otherwise indicated, all the numbers which express quantities, percentages or proportions, and other numerical values used in the specification and claims, are to be understood as being modified in all cases by the term “about.” Consequently, unless otherwise indicated, the numerical parameters presented in the following specification and appended claims are approximations that may vary depending on the desired properties sought to be obtained. At a minimum, each numerical parameter must be at least interpreted by considering the number of significant digits reported and by applying common rounding techniques.

### Examples

### Example 1-Design of a High-Affinity Artificial Anti-ZIKV Protein

The designed protein scaffold selected was based on cyclophilin B K163T from E. coli (PDB ID: 1J2A).26. The predicted structure is a 166-mer C-shaped globular protein, corresponding to SEQ ID NO: 2 (hereafter referred to as ZVPA3), composed of three β-sheets, two α-helix and three extended loops, providing a deep amphiphilic pocket into which the motif residues were initially grafted and the binding site created, as shown in FIG. 2 by the complementary surfaces. The pocket reaches the entire ZIKV FL region, using the three loops to significantly increase the contact area with the ZIKV FL. This is an important feature, as antibodies that target only parts of the ZIKV FL are not broadly neutralizing. After optimizing the interface, only two (of the four) native hydrophobic contacts were preserved, W101 (E)-Y112 (ZVPA3) and F108 (E)-Y53 (ZVPA3), suggesting that they play a central role in antibody-virus recognition (FIG. 2B). A total of 10 new interaction partners were created to increase binding affinity, as shown in FIG. 2B.

Several phenylalanine residues were initially present on the binding surface, either from the native sequence or assigned by the design algorithm developed. In order to increase the solubility of the protein without significantly affecting the binding affinity to the target and the propensity to tangle, most of the phenylalanine residues were replaced by tyrosine residues. Additional mutations were also introduced to stabilize the native folding. These mutations included the substitution of all cysteine residues in the core and on the surface of the protein with alanine and serine residues, respectively, to prevent aggregation. Five additional mutations, at least 8 A away from the binding site, were also reverted to their original residues in cyclophilin B K163T, as they had no impact on the calculated binding affinity. This strategy aimed to ensure adequate protein expression yields.

The final designed interface of ZVPA3 comprises 12 residues interacting with ZIKV E FL, 4 hydrogen bridges and 8 hydrophobic contacts (FIG. 2B).

### Example 2-Biophysical Characterization of ZVPA3

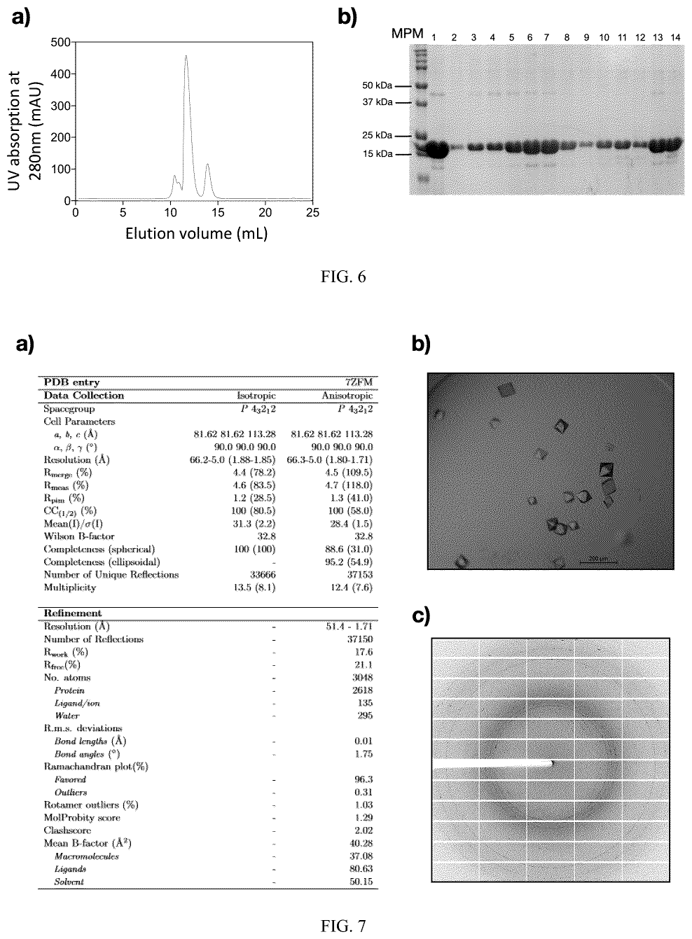

ZVPA3 was produced in E. coli as a monomer (ca. 20 kDa) and purified step by step using affinity chromatography and size exclusion (SEC) (FIG. 6).

To this effect, the DNA sequence encoding the computationally manipulated ZVPA3 was optimized for enhanced protein expression in a prokaryotic system, synthesized by GenScript and cloned into the expression vector pET14b (Novagen) with an N-terminal hexahistidine marker followed by a thrombin cleavage site. This expression vector was transformed in E. coli BL21 Star™ (DE3) pLysS One Shot™ (Thermo Fisher Scientific) by the standard heat shock procedure. The cells were grown in modified LB medium for self-induction (10 g/L NaCl, 10 g/L Tryptone, 5 g/L Yeast Extract, 0.125% (w/v) Glucose, 1.25% (w/v) Glycerol, 0.5% (w/v) alpha-Lactose, 0.12% (w/v) (NH₄)₂SO₄, 0.272% (w/v) KH₂PO₄, 0.284% (w/v) Na₂HPO₄ and 0.12% (w/v) MgSO₄) supplemented with carbenicillin (100 μg/mL) and chloramphenicol (34 μg/mL), incubated for 3 h at 37° C. and 16 h at 22° C. with orbital shaking at 200 rpm. The cells were harvested for 20 min at 7,000 g at 4° C. and resuspended in lysis buffer (50 mM Tris-Base pH 8.0, 300 mM NaCl, 20 mM Imidazole and 10% (w/v) Glycerol). The cells were ruptured at 15 kPsi (Constant Systems) and centrifuged at 10,000 g for 1 h, all at 4° C. The supernatant was loaded onto a 5 mL-HisTrap HP column (Cytiva) coupled to an HPLC system (ÄKTA Pure, Cytiva) and balanced with buffer A (50 mM Tris-base pH 8.0, 300 mM NaCl, 50 mM imidazole and 10% (w/v) glycerol). Elution was carried out with gradual 10% increases in buffer B (50 mM Tris-base pH 8.0, 300 mM NaCl, 300 mM imidazole and 10% (w/v) glycerol). The eluted fractions were dialyzed overnight in dialysis buffer (20 mM Tris-base pH 8.0 and 150 mM NaCl) and concentrated to 0.5 mL using Amicon R Ultra-4 Centrifugal Filter Unit-10,000 NMWL (Merck-Millipore). To further purify the sample, size exclusion chromatography was carried out using the Superdex 75 10/300 GL column (Cytiva) on the HPLC system. The fractions from the various purification steps were analyzed by SDS-PAGE with 15% polyacrylamide and Western blot using His-Probe (data not shown for conciseness). The selected fractions were pooled and concentrated to 11.6 mg/mL in 20 mM Tris pH 8 and 150 mM NaCl and subsequently used for biophysical and crystallization experiments. The protein yield was ca. 50 g/L of bacterial culture.

Next, the protein's secondary structure content and thermostability were evaluated by circular dichroism (CD) and differential scanning nanofluorimetry (nanoDSF), respectively.

The ZVPA3 samples were diluted from 1.25 to 0.25 mg/mL in 20 mM Tris pH 8 and 150 mM NaCl to select the best protein concentration. Subsequently, ZVPA3 at 0.25 mg/mL was diluted (˜50×) in different buffers (buffer 1-20 mM Tris-base pH 8.0, 150 mM NaCl; buffer 2-20 mM Tris-base pH 8.0; buffer 3-20 mM Tris-base pH 8.0, 150 mM NaF; buffer 4-20 mM Na Phosphate pH 8.0, 150 mM NaF; buffer 5-20 mM Na/Na Phosphate pH 8.0) to probe the secondary structure of the protein. CD spectra were measured on the JASCO J-815 spectropolarimeter (JASCO Inc, USA) from 190 to 260 nm, due to limitations in the voltage of the photomultiplier tubes (PMT), using a 0.1 cm path length quartz cuvette at 20° C. The CD spectra were acquired at scan rates of 50 nm/min with 3 accumulations and a response time of 4 s. Three independent replicates were carried out and merged. Spectral deconvolution was performed using the BestSel algorithm.^{47,48} Since some components of the buffers cause interference near the UV region of the spectrum, one was selected to estimate the secondary structure content of the protein.

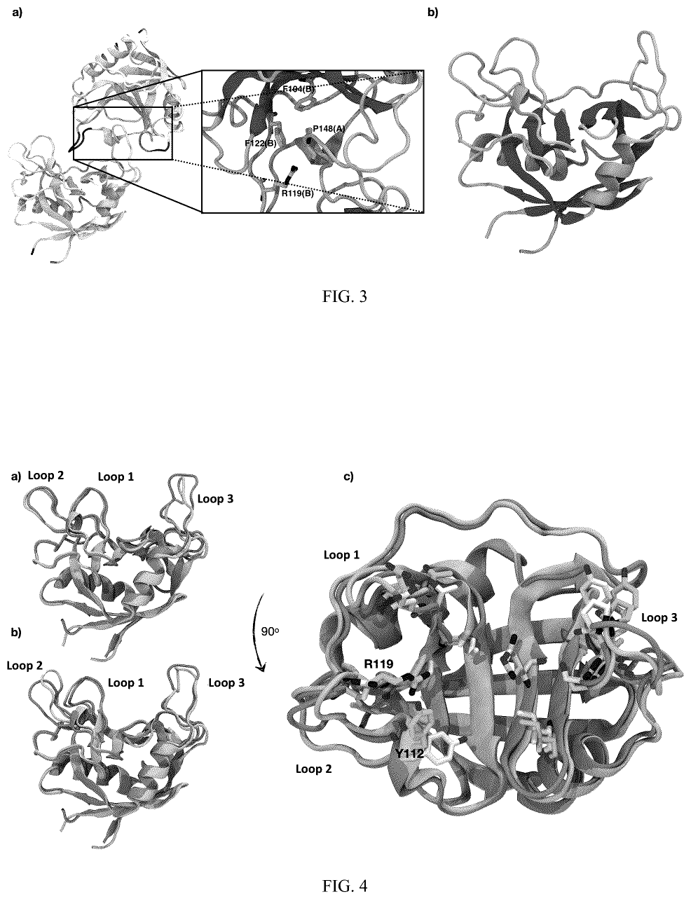

According to CD spectrum of the purified ZVPA3 recorded from 190 to 260 nm, the estimated secondary structure content of the protein comprises 14.8% of α-helix and 28.3% of β-sheets, which is in line with the calculated secondary structure content of the designed protein (15.1% of α-helix and 28.3% of β-sheets) (FIG. 3A).

NanoDSF experiments were carried out on a Prometheus NT.48 instrument (NanoTemper Technologies) to assess the thermostability of the designed protein. Samples of ZVPA3 at 1 mg/mL were diluted (˜11×) in various buffers (buffer 1-20 mM Tris-base pH 8.0, 150 mM NaCl; buffer 2-20 mM Tris-base pH 8.0; buffer 3-20 mM Tris-base pH 8.0, 150 mM NaF; buffer 4-20 mM Na Phosphate pH 8.0, 150 mM NaF; buffer 5-20 mM Na/Na Phosphate pH 8.0) and loaded into Prometheus glass capillaries (NanoTemper Technologies). The intrinsic fluorescence of the protein was recorded at wavelengths of 330 and 350 nm as the temperature increased from 20 to 120° C. at a rate of 1° C./min. Melting temperatures (Tₘ) were calculated from the minimum of the first derivative, corresponding to the temperature at which both the folded and unfolded states of the protein coexist in equilibrium.

The thermostability of the protein assessed by nanoDSF experiments showed ZVPA3 melting temperatures (Tₘ) between 48.1 and 50.4° C. (variations of less than 5% in all conditions tested) (FIG. 3B). In addition, the onset of the melting curve (Tₘ onset) related to the unfolding process was consistently around 42° C. Together, these data indicate that the ZVPA3 protein is adequately folded and stable in the buffers tested, thus allowing the crystallization experiments to proceed.

### Example 3-X-Ray Structure Determination for ZVPA3

Crystallization screens with ZVPA3 at 11.6 mg/mL in 20 mM Tris-base pH 8.0 and 150 mM NaCl were performed using commercial screens JSCG+, BCS, PACT Premier (Molecular Dimensions) in 3-well MRC plates (Molecular Dimensions) with the Mosquito LCP crystallization robot (SPT Labtech). Sitting-drops with final volumes of 200-300 nL were prepared by vapor diffusion using different protein:reservoir solution ratios (1:1, 1:2, 2:1), at 20° C. Octahedral-shaped crystals appeared after one week in 1.0 M succinic acid, 0.1 M Hepes pH 7.0 and 1% w/v PEG 2000 MME (JSCG+ condition #F11) and grew to approximately 250 μm3. The crystal diffracted to 1.71 A in the synchrotron source and belongs to the tetragonal space group P43212 with cell parameters a=b=81,618 and c=113,282 A (FIG. 7).

These crystals were cryoprotected in the crystallization condition supplemented with 25% glycerol or 25% ethylene glycol and immediately flash frozen in liquid nitrogen. X-ray diffraction data were measured at the XALOC beamline at the ALBA synchrotron (Barcelona, Spain) using the PILATUS 6M detector (Dectris) at a wavelength of 0.97926 Å. The data was indexed and integrated using XDS49, spatial group assigned with POINTLESS50 and scaled with SCALA/AIMLESS51 programs within the autoPROC.52 data processing pipeline. 5% of reflections were reserved for Rfree-flag. The ZVPA3 structure was solved by molecular replacement with Phaser53 within the PHENIX suite of programs,⁵⁴ using the E. coli cyclophilin B K163T structure without any solvent as a template (PDB accession number: 1J2A). The initial search was carried out with two molecules in the asymmetric unit according to the Matthews coefficient.²⁷ Model construction was performed with COOT55 and refinement with BUSTER-TNT56 and PHENIX suite⁵⁴ until convergence. The final coordinates have been deposited in the Protein Data Bank under the accession number 7ZFM.

The asymmetric crystal unit contains two molecules corresponding to a Matthews27 coefficient of 2.33 A3/Da and an approximate solvent content of 47.3%. The three-dimensional structure of ZVPA3 was solved by molecular replacement using the E. coli cyclophilin B K163T structure as a model [PDB code: 1J2A]0.26

The first 23 residues present in the designed plasmid, which corresponded to the N-terminal membrane-binding signal sequence (and are not part of the designed protein), were not visible in the electron density maps, likely due to self-cleavage during crystallization²⁸. The numbering of amino acid residues is done according to cyclophilin B, the template structure. Statistics on X-ray diffraction data collection/processing and crystallographic refinement are displayed in FIG. 7.

The electron density maps were generally well defined, except for loops G116 to G121 and H144 to P148 of the B chain, probably due to flexibility (FIG. 4A) and two bubbles of positive Fo-Fc electron density around both the alternative conformations of A-chain Y120. The ZVPA3 crystallographic model contains 330 amino acid residues (165 residues for each chain), 291 water molecules, two succinic acid ions, three acetate ions, one hexaethylene glycol, two triethylene glycol, and seventeen of 1,2-ethanediol. Furthermore, it is worth noting that the experimental structure has a glycine residue at the N- and C-terminus, which are not present in the computer-generated structure.

The crystallographic structure of ZVAP3 comprises two sets of four antiparallel β-sheets, in the shape of a slightly twisted β-barrel, flanked by two α-helix (FIG. 4B). Three flexible loops (Loop1: M91-S101, Loop2: D114-G121, Loop3: V141-P156) protrude from the protein core, forming a negatively charged pocket. This pocket has been described as the binding pocket of the CyPs proteins.²⁶ The asymmetric unit contains two ZVPA3 molecules, which are related by a rotation axis of almost a corkscrew fold (FIG. 4A). Analysis of the ZVPA3 crystallographic dimer by PISA29 showed a complex formation significance score (CSS) of 0, suggesting no physiological role for this oligomeric arrangement.

Molecules A and B intertwine their mobile loops with Loop3 of chain A, interacting with several residues in the binding pocket of chain B. Residues from sheets β3, β4 and β6 (chain A) make extensive contact with Loop3. P148 [A] coordinates two phenylalanine residues (F104 [B] and F122 [B]) and R119 [B] further stabilizes P148 [A] by hydrogen bonding with its carboxyl group. This increased stabilization is clearly shown by looking at the difference between the B factors of the same loop on both chains (FIG. 4A).

### Example 4-ZVPA3 Model Validation

The success of the modified protein depends on its ability to efficiently bind to its target, which is a direct consequence of its structure. CD experiments confirmed the amount of secondary structure content in the computer-generated model: however, as the desired function is a direct consequence of the predicted structure, this also requires confirmation. It is important to note that the model was designed in the presence of the antigen, while the crystal structure was designed in its absence. Furthermore, the experimental structure was determined in the presence of co-solutes (e.g., succinic acid and acetate ions, hexaethylene glycol, triethylene glycol, and seventeen molecules of 1,2-ethanediol).

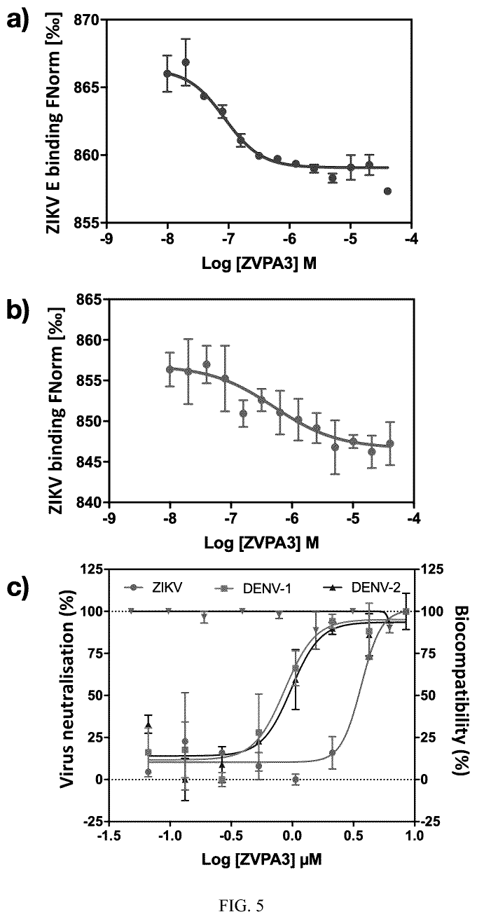

However, as disclosed above, the designed process aimed to design a stable protein, which was confirmed by the nanoDSF experiments. Therefore, no large-scale rearrangements in binding are anticipated. In fact, structural comparison between the computer-generated protein and the crystal structure reveals a striking similarity (FIG. 5). Structure superposition shows backbone RMSD values of 0.548 A and 0.639 A for chains A and B, respectively. The structural differences are confined to the terminal regions and around Loop3 for chain A (FIG. 5A) and Loop2 for chain B (FIG. 5B). These discrepancies are likely due to interactions observed between the A- and B-chain loops in the crystal cell unit (as reported above and shown graphically in FIG. 4A. Visual inspection of the side chain positions of the top 12 ZVPA3 residues interacting with ZIKV FL in the model (as shown in FIG. 2B), reveals that 10 of them have similar positional orientation in the crystal structure (FIG. 5B). In the absence of the binding target, the solvent-exposed residues in the Loop2 region, the Y112 and R119 side chains form a hydrogen bond (FIG. 5C). Otherwise, only small changes are observed for the remaining solvent-exposed residues, suggesting that ZVPA3 binding to ZIKV FL does not require large-scale induced docking movements.

### Example 5-Determination of ZIKV Binding Affinity and In Vitro Neutralization by ZVPA3

Cells and viruses. African green monkey kidney Vero cells (CCL-81, ATCC) were cultured at 37° C., 5% CO2 in Dulbecco's modified Eagle's medium (DMEM, Gibco) supplemented with 10% fetal bovine serum (FBS, Gibco), 100 units/mL penicillin/streptomycin (Gibco) and 0.25% amphotericin B (Gibco). The locally isolated ZIKV (strain PE243), DENV1 (PE/97-42735) and DENV2 (PE/95-3808), were propagated in Vero cells. Briefly, cells were grown to 80% confluence and virus isolates were inoculated at a multiplicity of infection (MOI) of 0.01 in DMEM medium supplemented with 2% FBS at 37° C. with gentle shaking for 2 h. Infected cells were incubated at 37° C., 5% CO2 until cytopathic effect (CPE) could be observed under a light microscope. Culture supernatants were harvested on day 6 by centrifugation at 400 g, 10 min, 4° C., aliquoted, and stored at −80° C. until use. Virus quantification was further determined using standard plaque assay. Briefly, culture supernatant containing ZIKV, and DENV1-2 isolates were serially diluted in DMEM supplemented with 2% FBS, 100 units/mL penicillin/streptomycin, and 0.25% amphotericin B and inoculated onto Vero cells at 37° C. with gentle stirring for 2 h. Supernatants were discarded and DMEM containing 1% (w/v) carboxymethyl cellulose was covered. Infected cells were incubated at 37° C., 5% CO2 until CPE was observed (6 days). The number of plaques was counted after fixing the cells with 10% formaldehyde and staining with 0.2% crystal violet.

The ultimate objective of the protein designed by ZVPA3 is the neutralization of the virus, which depends on a high-affinity interaction capable of preventing the virus from binding to cell surface receptors. It is important to highlight that the ZIKV E protein, the main target of neutralizing antibodies against ZIKV, exists in a dimeric form on the mature viral surface.³⁰ However, exposure of ZIKV antigens, including its FL, is driven by a phenomenon reported as viral respiration, in which the dynamic movement of the envelope continually samples different conformations.³¹ Thus, not all FL regions are available for binding. However, previous reports have shown that high affinity is a key determinant of an antibody's neutralizing activity in order to disrupt interactions between viral proteins and cellular receptors.³²⁻³⁴

Therefore, the affinity between ZVPA3 and the ZIKV E protein in its dimeric form was determined by microscale thermophoresis (MST) (FIG. 6A). The half-maximal binding parameter (EC₅₀) was determined from a non-linear regression analysis using a robust fit and revealed a dissociation constant (K_{D}) of 86.6 nM. The binding affinity for more than 10 ZIKV neutralizing antibodies or antibody fragments to the virus E protein has been determined experimentally. The value observed for ZVPA3 is within the same range found for other broadly neutralizing ZIKV antibodies targeting the E protein.^{25,35} Indeed, the binding affinities for these neutralizing mAbs vary widely from nM (e.g., 2A10G6 K_{D}=2.7 nM;²⁵ C8 K_{D}=9±1 nM; ¹⁶ ZK2β10 K_{D}=1.06 nM; ³⁶ ZV-48 K_{D}=35±0.8 nM; ¹⁷ZV-64 K_{D}=32±13 nM ¹⁷) to μM (e.g., A11 K_{D}=0.84±0.47 μM; ¹⁶ Z20 K_{D}=0.16 μM; ³⁷ Z23 K_{D}0.44 μM; ³⁷ Z3L1 K_{D}4.39 μM³⁷).

Twenty micromolars of commercially synthesized ZIKV Envelope (E) protein (Native Antigen, UK), or purified ZVPA3, were labeled using the fluorescent dye RED NT-647-NHS Labeling Kit (NanoTemper Technologies, Germany) according to the manufacturer's instructions. Unreacted dye was removed by buffer exchange chromatography (CE), while labeled proteins were eluted in 1× PBS buffer. The concentration of ZIKV E and ZVPA3 proteins was determined by spectrophotometry. The concentration of ZIKV infective particles was determined by the classical plaque assay and expressed as plaque forming units (PFU)/mL. Binding assays were performed using standard MST coated capillaries (NanoTemper). MST measurements were performed with 150 nM of labeled proteins (ZIKV E or ZVPA3) and a series of 16 serial 1:2 dilutions of the ligand in 1× PBS. 0.05% Tween-20 buffer. Measurements were performed at 298.15 K and 310.15 K using the Monolith NT.115 (NanoTemper). The red excitation LED was set to 40% and the laser power to medium. The laser on time was set to 30 s and the laser off time was set to 5 s. The quality of each MST run was assessed by performing a capillary scan before and after each run to verify that fluorescence between samples remained within 10% variation. The K_{D} of the interaction between ZVPA3 and the target epitope presented on the recombinant E protein was derived from three independent thermophoresis experiments using the law of mass action with NanoTemper MO. Affinity analysis software version 1.5.41. The EC₅₀ between ZVPA3 and the target epitope within the native viral particle was derived from three independent thermophoresis experiments and indicates apparent affinity by assuming that multiple copies of ZVPA3 would bind to the same ZIKV particle. The resulting binding curves were plotted using Prism 7.0 software.

It is possible to estimate an approximate stoichiometry for the ZVPA3:E_{(Dimer)} from the MST data. From test concentrations, maximum binding is observed when ca. 3×10¹³ dimer E molecules are in the presence of ca. 8×10¹² ZVPA3 molecules. Although a stoichiometry of 2:1 ZVPA3:E_{(Dimer)} can be expected, since each E dimer has two FL, the above data provides a ratio of 1:3,75 ZVPA3:E_{(Dimer)}. The lower-than-expected stoichiometry is due to the fact that not all FL sites will be exposed in the E protein. To obtain information about the stability of the ZVPA-E_{(Dimer)} complex, the thermal unfolding of the complex was measured, at the estimated stoichiometry, and compared only with ZVPA3.

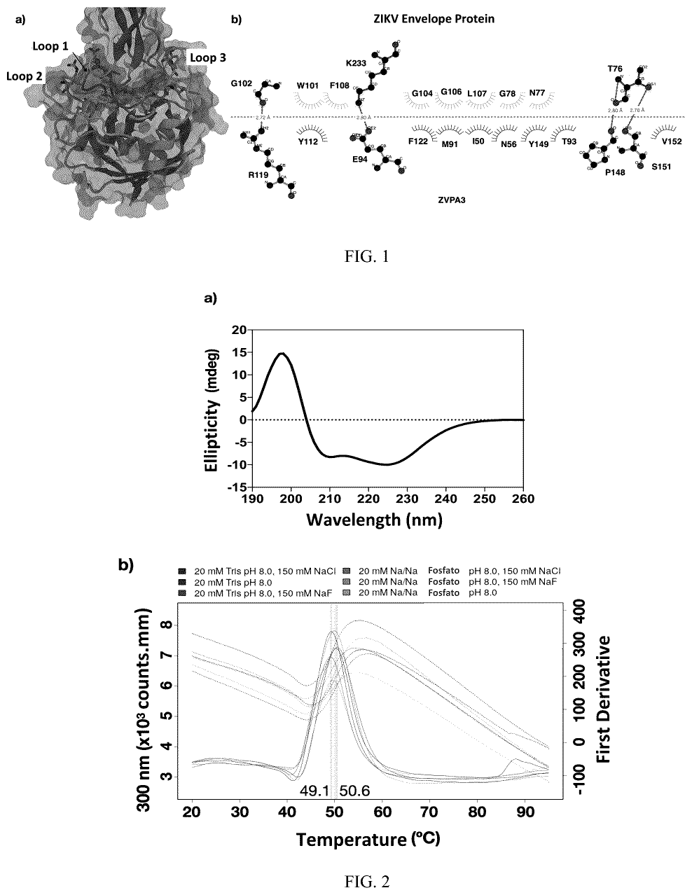

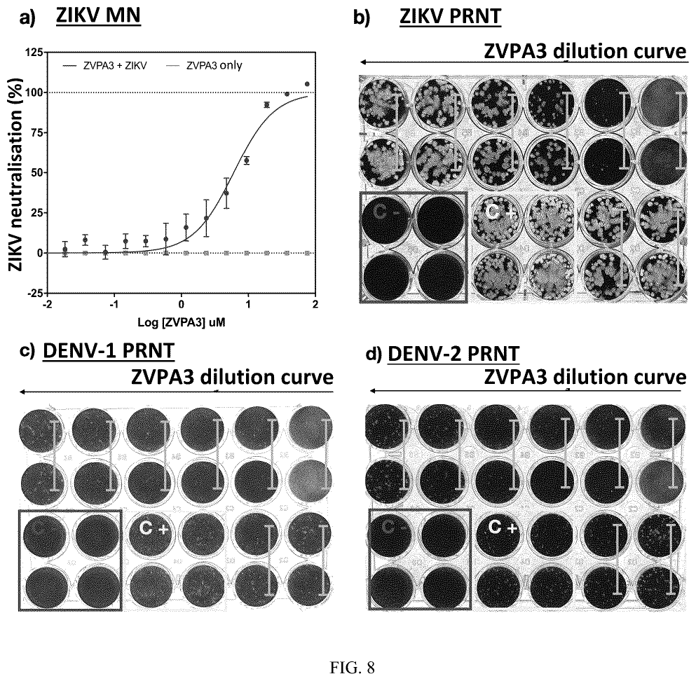

A stability gain of ca. 5° C. was observed for the complex (FIG. 8). Greater stability for the ZVPA3 complex is expected from the designed surface. FIG. 1B shows that the contacts between ZIKV E and the designed pocket of ZVPA3 are primarily hydrophobic in nature. Although steric factors, hydrophobic and electrostatic interactions, including hydrogen bonds, contribute to binding, hydrophobic forces have been shown to play a more significant role in the stability of protein interactions.³⁸

However, neutralization is ultimately a function of epitope accessibility in the context of the entire virus. During the maturation process of flaviviruses, the E DII protein binds to the premembrane protein (prM), which is cleaved by a cellular furin during the transport of viral particles through the secretory pathway. The result is a mature virus particle featuring a compact E-dimer arrangement that holds ZIKV FL in a hidden conformation buried in a cleft composed of domains I and III of the other E monomers. 25 Therefore, some hidden epitopes for neutralizing antibodies can only be exposed to antibody recognition during the cellular infection process. Therefore, the ability of a protein to bind (i.e. binding affinity) should not be expected to be comparable to the protein isolated in solution.

During this phase, pH and temperature transitions (above 34° C.) lead to conformational changes in the virus particle, as has been demonstrated for DENV.^{39,40} and prM cleavage occurs, momentarily exposing ZIKV FL. Taking this information into consideration, additional MST experiments were performed using ZVPA3 and the entire ZIKV particle at physiological temperature (37° C.) (FIG. 6B). The binding affinity of ZVPA3 for the target epitope across the virus showed a K_{D} of 0.507 μM. The same quantity for 2A10G6 is not available for comparison.

After determining the target binding affinity, the flavivirus neutralization activity of ZVPA3 was determined in vitro using the plaque reduction neutralization test (PRNT) against locally isolated strains of ZIKV and DENV1-2 (FIG. 6C). Determination of flavivirus neutralizing activity through the plaque reduction neutralization test (PRNT) and microneutralization test (MN). The evaluation of the virus-specific neutralizing activity of the designed protein ZVPA3 was performed by the microneutralization test (MN) and the plaque reduction neutralization test (PRNT). While the latter relies on visual plaque counts, MN measures virus neutralization potency using a colorimetric readout. Neutralization of ZIKV and DENV-1/2 was assessed using PRNT following a protocol described in detail elsewhere.⁵⁷ Neutralization was assessed against the following locally isolated virus strains: ZIKV (BR-PE243/2015), DENV1 (PE/97-42735) and DENV2 (PE/95-3808). The half maximum effective concentration (EC₅₀) of ZVPA3 against ZIKV and DENV-1/2 was determined by calculating the protein dilution that neutralized 50% of the maximum signal using a four-parameter nonlinear regression (Graphpad Prism v.7 software).

MN was performed only to confirm ZIKV neutralizing activity. For this assay, double serial dilutions of the designed protein were performed using 50 μL aliquots in the rows of a 96-well plate. Next, 625 PFU/mL of the ZIKV challenge dose (in 50 μL) were mixed with the serially diluted protein and left to neutralize the infectivity of the virus for 2 h at 37° C. After neutralization, an aliquot of 100 μL containing 80,000 cells of a fresh suspension of Vero cells was added to each well of the plates. The plates were incubated for 72 h at 37° C. with 5% CO2 and humidity. The plates were washed with 1× PBS supplemented with 0.1% (v/v) Tween-20 (PBS-T) and fixed with 80% acetone, followed by blocking with 5% (w/v) skimmed milk (BIO-RAD) in PBS-T for 30 min at room temperature and incubated with cross-reacting monoclonal antibody (anti-Flavivirus 4G2 mAb at 0.6 μg/mL) for 2 h at room temperature. After the washing step, the plates were incubated with horseradish-conjugated goat anti-mouse IgG (Sigma) at a dilution of 1:2,500 for 1 hour at room temperature. The plates were washed and developed with TMB-KPL microwell peroxidase substrate system (Pierce, IL, USA) for 45 min at room temperature. The reaction was stopped with 1 M hydrochloric acid and the absorbance at 450 nm was measured on a BioTek microplate reader (Winooski, VT, USA). The EC₅₀ of ZVPA3 against ZIKV was confirmed by calculating the dilution of the protein that neutralized 50% of the maximum signal using a four-parameter non-linear regression (Graphpad Prism v.7 software). Uninfected and ZIKV- or DENV-infected Vero cells were included in each plate and used as negative and positive controls, respectively. The absorbance of the positive control wells was used to calculate the reduction in virus infectivity by ZVPA3, as these wells represented 100% virus infectivity (or 0% virus neutralization). A virus titration curve was included on each plate to ensure the reproducibility of the assay.

The present data show that ZVPA3 recognized ZIKV in the context of cellular infection and efficiently reduced ZIKV infection in vitro (compared to assay controls) with a half-maximal binding parameter (EC₅₀) of 3.65 μM. ZIKV neutralization was also confirmed by a complementary technique (in vitro microneutralization test) and showed a similar EC₅₀ value (6.06 μM, Figure support S3). Conversion of the PRNT₅₀ neutralization of 2A10G6 to ZIKV of 249 μg/mL gives an EC₅₀ of 1.66 μM. Interestingly, despite the higher affinity of the antibody for the ZIKV E protein isolated in solution, a similar neutralization potential of ZIKV in vitro is observed for 2A10G6 and ZVPA3. In addition, ZVPA3 also neutralized DENV1 and DENV2 infection in vitro with statistically similar efficiency, exhibiting EC₅₀ values of 0.87 and 0.98 μM, respectively (FIG. 6C). This would be expected given the conserved 98DRGW101 sequence of the FL between these flaviviruses. In addition, biocompatibility tests show that the ZVPA3 protein did not show cell toxicity in vitro. At the highest concentration used in this assay, a reduction of only 9.86% was observed in the cell biocompatibility measure (shown on the y-axis to the right of FIG. 6C).

It is worth noting that antibodies targeting ZIKV FL are generally linked to antibody-dependent enhancement (ADE) while exhibiting limited neutralizing activity.^{23,37,41} Here. ZVPA3 has been shown to exhibit comparable cross-reactive neutralizing potency against ZIKV and DENV1-2 in vitro compared to the broadly neutralizing antibody 2A10G6 (PRNT₅₀/EC₅₀ of 249 μg/mL or 1.66 μM), whose interaction interface has been used as an inspiring starting point for protein design.²⁵ The virus neutralization mechanism for ZVPA3 is believed to be based on the stability of the manipulated protein interface for the ZIKV FL epitope. This is supported by the positional agreement of even the side chains of Loops 1 and 2, as well as other predicted interacting residues, when comparing the crystal structure in the absence of the ligand with the designed structure, which was designed in the presence of the antigen. The residues in Loop 3 of the designed structure did not overlap well with the crystal counterpart.

Despite this, the protein shows high binding affinity to its target and excellent EC₅₀ neutralization. Taking everything into account, it suggests that only a small, induced fit is required for binding.

Moreover, it is important to mention that ZVPA3 was computationally designed as a small protein that carries only a new paratope for virus recognition. Therefore, the Fc portion of the antibody (necessary for ADE to occur) is not present in the ZVPA3 molecule. Furthermore, antibody-mediated neutralization requires the engagement of virions by antibodies with a sufficient stoichiometry for recognition.¹⁷ Therefore, the size of ZVPA3 is believed to be more suitable for viral neutralization than conventional antibodies.

### REFERENCES

1. M. R. Holbrook, Viruses, 2017, 9.
2. M. G. Guzman and E. Harris, Lancet, 2015, 385, 453-465.
3. M. S. Suthar, M. S. Diamond and M. Gale, Jr., Nat Rev Microbiol, 2013, 11, 115-128.
4. T. P. Monath and P. F. Vasconcelos, J Clin Virol, 2015, 64, 160-173.
5. D. Baud, D. Musso, M. Vouga, M. P. Alves and N. Vulliemoz, Am J Reprod Immunol, 2017, 77.
6. O. Faye, C. C. Freire, A. Iamarino, O. Faye, J. V. de Oliveira, M. Diallo, P. M. Zanotto and A. A. Sall, PLOS Negl Trop Dis, 2014, 8, e2636.
7. J. Mlakar, M. Korva, N. Tul, M. Popovic, M. Poljsak-Prijatelj, J. Mraz, M. Kolenc, K. Resman Rus, T. Vesnaver Vipotnik, V. Fabjan Vodusek, A. Vizjak, J. Pizem, M. Petrovec and T. Avsic Zupanc, The New England journal of medicine, 2016, 374, 951-958.
8. T. V. B. de Araujo, R. A. A. Ximenes, D. B. Miranda-Filho, W. V. Souza, U. R. Montarroyos, A. P. L. de Melo, S. Valongueiro, M. de Albuquerque, C. Braga, S. P. B. Filho, M. T. Cordeiro, E. Vazquez, D. Cruz, C. M. P. Henriques, L. C. A. Bezerra, P. Castanha, R. Dhalia, E. T. A. Marques-Junior, C. M. T. Martelli, L. C. Rodrigues, G. investigators from the Microcephaly Epidemic Research, H. Brazilian Ministry of, O. Pan American Health, F. Instituto de Medicina Integral Professor Fernando and P. State Health Department of, Lancet Infect Dis, 2018, 18, 328-336.
9. V. M. Cao-Lormeau, A. Blake, S. Mons, S. Lastere, C. Roche, J. Vanhomwegen, T. Dub, L. Baudouin, A. Teissier, P. Larre, A. L. Vial, C. Decam, V. Choumet, S. K. Halstead, H. J. Willison, L. Musset, J. C. Manuguerra, P. Despres, E. Fournier, H. P. Mallet, D. Musso, A. Fontanet, J. Neil and F. Ghawche, Lancet, 2016, 387, 1531-1539.
10. G. Paz-Bailey, E. S. Rosenberg, K. Doyle, J. Munoz-Jordan, G. A. Santiago, L. Klein, J. Perez-Padilla, F. A. Medina, S. H. Waterman, C. G. Gubern, L. I. Alvarado and T. M. Sharp, The New England journal of medicine, 2018, 379, 1234-1243.
11. B. D. Lindenbach and C. M. Rice, Adv Virus Res, 2003, 59, 23-61.
12. V. A. Kostyuchenko, E. X. Lim, S. Zhang, G. Fibriansah, T. S. Ng, J. S. Ooi, J. Shi and S. M. Lok, Nature, 2016, 533, 425-428.
13. D. Sirohi, Z. Chen, L. Sun, T. Klose, T. C. Pierson, M. G. Rossmann and R. J. Kuhn, Science, 2016, 352, 467-470.
14. Y. Zhang, W. Zhang. S. Ogata, D. Clements, J. H. Strauss, T. S. Baker, R. J. Kuhn and M. G. Rossmann, Structure, 2004, 12, 1607-1618.
15. C. Lopez-Camacho, G. De Lorenzo, J. L. Slon-Campos, S. Dowall, P. Abbink, R. A. Larocca, Y. C. Kim, M. Poggianella, V. Graham, S. Findlay-Wilson, E. Rayner, J. Carmichael, W. Dejnirattisai, M. Boyd, R. Hewson, J. Mongkolsapaya, G. R. Screaton, D. H. Barouch, O. R. Burrone, A. H. Patel and A. Reyes-Sandoval, Vaccines (Basel), 2020, 8.
16. G. Barba-Spaeth, W. Dejnirattisai, A. Rouvinski, M. C. Vaney, I. Medits, A. Sharma, E. Simon-Loriere, A. Sakuntabhai, V. M. Cao-Lormeau, A. Haouz, P. England, K. Stiasny, J. Mongkolsapaya, F. X. Heinz, G. R. Screaton and F. A. Rey, Nature, 2016, DOI: 10.1038/nature18938.
17. H. Zhao, E. Fernandez, K. A. Dowd, S. D. Speer, D. J. Platt, M. J. Gorman, J. Govero, C. A. Nelson, T. C. Pierson, M. S. Diamond and D. H. Fremont, Cell, 2016, 166, 1016-1027.
18. M. Beltramello, K. L. Williams, C. P. Simmons, A. Macagno, L. Simonelli, N. T. Quyen, S. Sukupolvi-Petty, E. Navarro-Sanchez, P. R. Young, A. M. de Silva, F. A. Rey, L. Varani, S. S. Whitehead, M. S. Diamond, E. Harris, A. Lanzavecchia and F. Sallusto, Cell Host Microbe, 2010, 8, 271-283.
19. Y. Q. Deng, J. X. Dai, G. H. Ji, T. Jiang, H. J. Wang, H. O. Yang, W. L. Tan, R. Liu, M. Yu, B. X. Ge, Q. Y. Zhu, E. D. Qin, Y. J. Guo and C. F. Qin, PloS one, 2011, 6, e16059.
20. C. Y. Lai, K. L. Williams, Y. C. Wu, S. Knight, A. Balmaseda, E. Harris and W. K. Wang, PLOS Negl Trop Dis, 2013, 7, e2451.
21. M. R. Vogt, K. A. Dowd, M. Engle, R. B. Tesh, S. Johnson, T. C. Pierson and M. S. Diamond, Journal of virology, 2011, 85, 11567-11580.
22. J. C. Frei, A. S. Wirchnianski, J. Govero, O. Vergnolle, K. A. Dowd, T. C. Pierson, M. Kielian, M. E. Girvin, M. S. Diamond and J. R. Lai, Journal of virology, 2018, 92.
23. W. Dejnirattisai, W. Wongwiwat, S. Supasa, X. Zhang, X. Dai, A. Rouvinski, A. Jumnainsong, C. Edwards, N. T. H. Quyen, T. Duangchinda, J. M. Grimes, W. Y. Tsai, C. Y. Lai, W. K. Wang, P. Malasit, J. Farrar, C. P. Simmons, Z. H. Zhou, F. A. Rey, J. Mongkolsapaya and G. R. Screaton, Nature immunology, 2015, 16, 170-177.
24. W. Y. Tsai, C. Y. Lai, Y. C. Wu, H. E. Lin, C. Edwards, A. Jumnainsong, S. Kliks, S. Halstead, J. Mongkolsapaya, G. R. Screaton and W. K. Wang, Journal of virology, 2013, 87, 12562-12575.
25. L. Dai, J. Song, X. Lu, Y. Q. Deng, A. M. Musyoki, H. Cheng, Y. Zhang, Y. Yuan, H. Song, J. Haywood, H. Xiao, J. Yan, Y. Shi, C. F. Qin, J. Qi and G. F. Gao, Cell Host Microbe, 2016, 19, 696-704.
26. M. Konno, Y. Sano, K. Okudaira, Y. Kawaguchi, Y. Yamagishi-Ohmori, S. Fushinobu and H. Matsuzawa, Eur J Biochem, 2004, 271, 3794-3803.
27. B. W. Matthews, Journal of molecular biology, 1968, 33, 491-497.
28. P. H. Hirel, M. J. Schmitter, P. Dessen, G. Fayat and S. Blanquet, Proceedings of the National Academy of Sciences of the United States of America, 1989, 86, 8247-8251.
29. E. Krissinel and K. Henrick, Journal of molecular biology, 2007, 372, 774-797.
30. C. Yang, F. Zeng, X. Gao, S. Zhao, X. Li, S. Liu, N. Li, C. Deng, B. Zhang and R. Gong, J Biol Chem, 2019, 294, 10638-10648.
31. R. J. Kuhn, K. A. Dowd, C. Beth Post and T. C. Pierson, Virology, 2015, 479-480, 508-517.
32. G. Bauer, Int J Infect Dis, 2021, 106, 61-64.
33. I. Khatri, F. J. T. Staal and J. J. M. van Dongen, Front Immunol, 2020, 11, 570018.
34. S. Fiedler, M. A. Piziorska, V. Denninger, A. S. Morgunov, A. Ilsley, A. Y. Malik, M. M. Schneider, S. R. A. Devenish, G. Meisl, V. Kosmoliaptsis, A. Aguzzi, H. Fiegler and T. P. J. Knowles, ACS Infect Dis, 2021, 7, 2362-2369.
35. P. Qu, C. Zhang, M. Li, W. Ma, P. Xiong, Q. Liu, G. Zou, D. Lavillette, F. Yin, X. Jin and Z. Huang, Cell Discov, 2020, 6, 5.
36. L. Wang, R. Wang, L. Wang, H. Ben, L. Yu, F. Gao, X. Shi, C. Yin, F. Zhang, Y. Xiang and L. Zhang, Cell Rep, 2019, 26, 3360-3368 e3365.
37. Q. Wang, H. Yang, X. Liu, L. Dai, T. Ma, J. Qi, G. Wong, R. Peng, S. Liu, J. Li, S. Li, J. Song, J. Liu, J. He, H. Yuan, Y. Xiong, Y. Liao, J. Li, J. Yang, Z. Tong, B. D. Griffin, Y. Bi, M. Liang, X. Xu, C. Qin, G. Cheng, X. Zhang, P. Wang, X. Qiu, G. Kobinger, Y. Shi, J. Yan and G. F. Gao, Sci Transl Med, 2016, 8, 369ral79.
38. S. Jones and J. M. Thornton, Proc Natl Acad Sci USA, 1996, 93, 13-20.
39. G. Fibriansah, T. S. Ng, V. A. Kostyuchenko, J. Lee, S. Lee, J. Wang and S. M. Lok, Journal of virology, 2013, 87, 7585-7592.
40. X. Zhang, P. Ge, X. Yu, J. M. Brannan, G. Bi, Q. Zhang, S. Schein and Z. H. Zhou, Nature structural & molecular biology, 2013, 20, 105-110.
41. K. Stettler, M. Beltramello, D. A. Espinosa, V. Graham, A. Cassotta, S. Bianchi, F. Vanzetta, A. Minola, S. Jaconi, F. Mele, M. Foglierini, M. Pedotti, L.

Simonelli, S. Dowall, B. Atkinson, E. Percivalle, C. P. Simmons, L. Varani, J. Blum, F. Baldanti, E. Cameroni, R. Hewson, E. Harris, A. Lanzavecchia, F. Sallusto and D. Corti, Science, 2016, 353, 823-826.
42. L. Dai, J. Song, X. Lu, Y.-Q. Deng, Abednego M. Musyoki, H. Cheng, Y. Zhang, Y. Yuan, H. Song, J. Haywood, H. Xiao, J. Yan, Y. Shi, C.-F. Qin, J. Qi and George F. Gao, Cell Host & Microbe, 2016, 19, 696-704.
43. A. Leaver-Fay, M. Tyka, S. M. Lewis, O. F. Lange, J. Thompson, R. Jacak, K. W. Kaufman, P. D. Renfrew, C. A. Smith, W. Sheffler, I. W. Davis, S. Cooper, A. Treuille, D. J. Mandell, F. Richter, Y.-E. A. Ban, S. J. Fleishman, J. E. Corn, D. E. Kim, S. Lyskov, M. Berrondo, S. Mentzer, Z. Popović, J. J. Havranek, J. Karanicolas, R. Das, J. Meiler, T. Kortemme, J. J. Gray, B. Kuhlman, D. Baker and P. Bradley, in Meth. Enzymol., eds. M. L. Johnson and L. Brand, Academic Press, 2011, vol. 487, pp. 545-574.
44. S. J. Fleishman and D. Baker, Cell, 2012, 149, 262-273.
45. S. J. Fleishman, A. Leaver-Fay, J. E. Corn, E.-M. Strauch, S. D. Khare, N. Koga, J. Ashworth, P. Murphy, F. Richter, G. Lemmon, J. Meiler and D. Baker, PLOS One, 2011, 6, e20161.
46. R. F. Alford, A. Leaver-Fay, J. R. Jeliazkov, M. J. O'Meara, F. P. DiMaio, H. Park, M. V. Shapovalov, P. D. Renfrew, V. K. Mulligan, K. Kappel, J. W. Labonte, M. S. Pacella, R. Bonneau, P. Bradley, R. L. Dunbrack, R. Das, D. Baker, B. Kuhlman, T. Kortemme and J. J. Gray, J. Chem. Theory Comput., 2017, 13, 3031-3048.
47. A. Micsonai, F. Wien, E. Bulyaki, J. Kun, E. Moussong, Y. H. Lee, Y. Goto, M. Refregiers and J. Kardos, Nucleic acids research, 2018, 46, W315-W322.
48. A. Micsonai, F. Wien, L. Kemnya, Y. H. Lee, Y. Goto, M. Refregiers and J. Kardos, Proceedings of the National Academy of Sciences of the United States of America, 2015, 112, E3095-3103.
49. W. Kabsch, Acta Crystallogr D Biol Crystallogr, 2010, 66, 125-132.
50. P. Evans, Acta Crystallogr D Biol Crystallogr, 2006, 62, 72-82.
51. P. R. Evans and G. N. Murshudov, Acta Crystallogr D Biol Crystallogr, 2013, 69, 1204-1214.
52. C. Vonrhein, C. Flensburg, P. Keller, A. Sharff, O. Smart, W. Paciorek, T. Womack and G. Bricogne, Acta Crystallogr D Biol Crystallogr, 2011, 67, 293-302.
53. A. J. McCoy, Acta Crystallogr D Biol Crystallogr, 2007, 63, 32-41.
54. P. D. Adams, P. V. Afonine, G. Bunkoczi, V. B. Chen, I. W. Davis, N. Echols, J. J. Headd, L. W. Hung, G. J. Kapral, R. W. Grosse-Kunstleve, A. J. McCoy, N. W. Moriarty, R. Oeffner, R. J. Read, D. C. Richardson, J. S. Richardson, T. C. Terwilliger and P. H. Zwart, Acta Crystallogr D Biol Crystallogr, 2010, 66, 213-221.
55. P. Emsley, B. Lohkamp, W. G. Scott and K. Cowtan, Acta Crystallogr D Biol Crystallogr, 2010, 66, 486-501.
56. E. Blanc, P. Roversi, C. Vonrhein, C. Flensburg, S. M. Lea and G. Bricogne, Acta Crystallogr D Biol Crystallogr, 2004, 60, 2210-2221.
57. E. J. M. Nascimento, M. I. Bonaparte, P. Luo, T. S. Vincent, B. Hu, J. K. George, G. Anez, F. Noriega, L. Zheng and J. W. Huleatt, Am J Trop Med Hyg, 2019, 101, 708-715.
58. R. K. A. d. O. França; J. M. Silva; L. S. Rodrigues; D. Sokolowskei; M. M. Brigido; A. Q. Maranhão, New Anti-Flavivirus Fusion Loop Human Antibodies with Zika Virus-Neutralizing Potential. Int. J. Mol. Sci. 2022, 23, 7805. https://doi.org/10.3390/ijms23147805.

1. Polynucleotide, characterized in that it is selected from the group consisting of:
(a) polynucleotides comprising a nucleotides sequence as set out in SEQ ID NO: 1;
(b) nucleic acids that hybridize under stringent conditions with the nucleic acid of SEQ ID NO: 1;
(c) polynucleotides comprising a nucleic acid sequence having at least 70% identity with the sequence as set out in SEQ ID NO: 1;
(d) polynucleotides that encode a polypeptide that is at least 70% identical to the amino acid sequence of SEQ ID NO: 2 or 3;
(e) degenerated sequences of polynucleotides from (a)-(d).

2. Polynucleotide according to claim 1, characterized in that it is a cDNA, genomic DNA, synthetic DNA or RNA.

3. Expression cassette, characterized in that it comprises a polynucleotide as defined in claim 1 operably linked to a promotor and to a transcription terminator.

4. Expression vector, characterized in that it comprises a polynucleotide as defined in claim 1.

5. Host cell, characterized in that it comprises the polynucleotide as defined in claim 1.

6. Polypeptide, characterized in that it is selected from the group consisting of:
(a) a polypeptide comprising the amino acid sequence as set out in SEQ ID NO: 2 or 3; or
(b) a polypeptide comprising an amino acid sequence having at least 70% identity with the sequence as set out in SEQ ID NO: 2 or 3;
in its pure form, or fused or complexed with nanomaterials, hydrogels, other proteins, liposomes and any other carrier and/or release vehicle.

7. Pharmaceutical composition, characterized in that it comprises a polypeptide as defined in claim 6, and a pharmaceutically acceptable carrier or excipient.

8. Composition according to claim 7, characterized in that said composition is a medicine, a vaccine or a therapeutic vaccine.

9. Polypeptide according to claim 6, characterized in that it is for the prevention or treatment of infections caused by flaviviruses.

10. Polypeptide according to claim 9, characterized in that the flavivirus are selected from Zika virus (ZIKV) or Dengue virus (DENV-1, DENV-2).

11. Use of a polypeptide as defined in claim 6, characterized in that it is in the manufacture of a medicine for the prevention or treatment of infections caused by flaviviruses.

12. Use according to claim 11, characterized in that the flavivirus are selected from Zika virus (ZIKV) or Dengue virus (DENV-1, DENV-2).

13. Method for producing a polypeptide, characterized in that it comprises:
(a) providing a host cell as defined in claim 5;
(b) culturing said cell under conditions conducive to the production of the polypeptide; and
(c) isolating said polypeptide from said cell or the culture medium in which it is inserted.

14. Method for preventing or treating infections caused by flaviviruses, characterized in that it comprises administering a therapeutically effective amount of the polypeptide as defined in claim 6 to a subject in need thereof.

15. Method according to claim 14, characterized in that the flavivirus are selected from Zika virus (ZIKV) or Dengue virus (DENV-1, DENV-2).


SEQUENCE LISTING (3 sequences)

SEQ ID NO: 1 - DNA, 576 bp, synthetic construct
  source 1..576: mol_type = other DNA
atgggcagcagccatcatcatcatcatcacagcagcggcctggtgccgcgcggcagccatatggcgaaaggcgacccgcacgtgctgctgaccaccagcgcgggtaacatcgagctggaactggacagccagaaagcgccggtgagcgttcaaaacttcctggattacgtgaacaacggcttctataacaacaccacctttcacagcgttatcccgggttacatgattcagggtggcggttataccgaggacatgcagcaaaagcaaccgaacccgccgatcaaaaacgaagcggataacggcctgcgtaacacccgtggtaccattgcgatgatgcgtaccgaggacaaggatagcgcgaccagccagttctttattaacgttgcggacaacgcgtacgcggatcacggccaaacccgttatggtttcgcggtgtttggcaaggtggttaagggtatggacgttgcggataagatcagccaggtgccgacccacgatgttggcccgtaccaaagcgtgccgagcaaaccggtggttattctgagcgcgaccgttctgccgggatccggctgctaa

SEQ ID NO: 2 - protein, 166 aa, synthetic construct
AKGDPHVLLTTSAGNIELELDSQKAPVSVQNFLDYVNNGFYNNTTFHSVIPGYMIQGGGYTEDMQQKQPNPPIKNEADNGLRNTRGTIAMMRTEDKDSATSQFFINVADNAYADHGQTRYGFAVFGKVVKGMDVADKISQVPTHDVGPYQSVPSKPVVILSATVLP

SEQ ID NO: 3 - protein, 191 aa, synthetic construct
MGSSHHHHHHSSGLVPRGSHMAKGDPHVLLTTSAGNIELELDSQKAPVSVQNFLDYVNNGFYNNTTFHSVIPGYMIQGGGYTEDMQQKQPNPPIKNEADNGLRNTRGTIAMMRTEDKDSATSQFFINVADNAYADHGQTRYGFAVFGKVVKGMDVADKISQVPTHDVGPYQSVPSKPVVILSATVLPGSGC